(19) United States
(12) Patent Application Publication
(10) Pub. No.: US 2006/0046994 A1
(43) Pub. Date: Mar. 2, 2006

(54) Process for preparing 3-oxo-4-aza-5-alpha-androstane-17-carboxylic acid

(76) Inventors: Sundaram Venkataraman, Hyderabad (IN); Srinivasulu Godipati, Hyderabad (IN); Mayur Devjibhai Khunt, Junagadh (IN); Ramchandra Reddy Bojja, Cuddapah (IN); Naveenkumar Reddy Keshava, Hyderabad (IN); Somaiah Sripathi, Nalgonda (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/210,528

(22) Filed: Aug. 24, 2005

Related U.S. Application Data
(60) Provisional application No. 60/604,264, filed on Aug. 25, 2004.

(51) Int. Cl. C07D 221/18; A61K 31/473

(52) U.S. Cl. 514/294; 546/61

(57) ABSTRACT

A process for preparing the compound 3-oxo-4-aza-5α-androstane-17β-carboxylic acid having Formula (I): 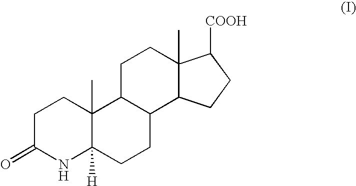 which is an intermediate in the preparation of 4-azasteroid drug compounds like finesteride, dutasteride, etc.

### INTRODUCTION TO THE INVENTION

The present invention relates to a simple process for the preparation of 3-oxo-4-aza-5α-androstane-17β-carboxylic acid (Formula (I)), which is an intermediate for preparing 4-azasteroid drug compounds like finesteride, dutasteride, etc.

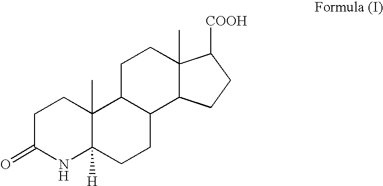

The general formula of the azasteroid drug compounds can be depicted by Formula (Ia). 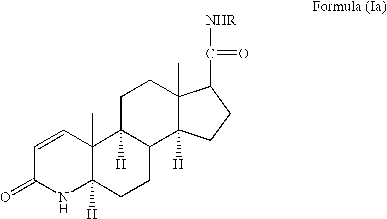
wherein R is 2,5-bis-(trifluoromethyl)phenyl,1-dimethylethyl, etc. The azasteroid compounds are useful as specific inhibitors of steroid Type II 5α-reductase, an intracellular enzyme that converts the androgen testosterone into 5α-dihydrotestosterone.

U.S. Pat. No. 4,760,071 discloses a process for preparing 3-Oxo-4-aza-5-etian-20-oic acid which involves cyclizing 5-Oxo-3,5-secoetian-3,20-dioic acid in ethylene glycol using liquid ammonia and heating to 180° C. The patent also discloses the preparation of 3-Oxo-4-aza-5α-etian-20-oic acid by hydrogenating 3-Oxo-4-aza-5-etien-20-oic acid using a platinum catalyst in the presence of acetic acid.

G. H. Rasmusson et. al., Journal of Medicinal Chemistry, Vol. 27, pages 1690-1701, 1984 disclosed oxidation of a compound of the present Formula (II) to the present Formula (III) by using sodium metaperiodate and potassium permanganate in 2-methylpropan-2-ol.

According to an article titled “Finasteride (Propecia®)” posted on Feb. 3, 1998 at the website:

http://www.phc.vcu.edu/Feature/oldfeature/finasteride/finasteride2.html by Cynthia L. Schieck, G. H. Rasmusson et al., Journal of Medicinal Chemistry, Vol. 29, pages 2298-2315, 1986 described a synthesis of finasteride, a portion of which proceeds according to the following scheme: 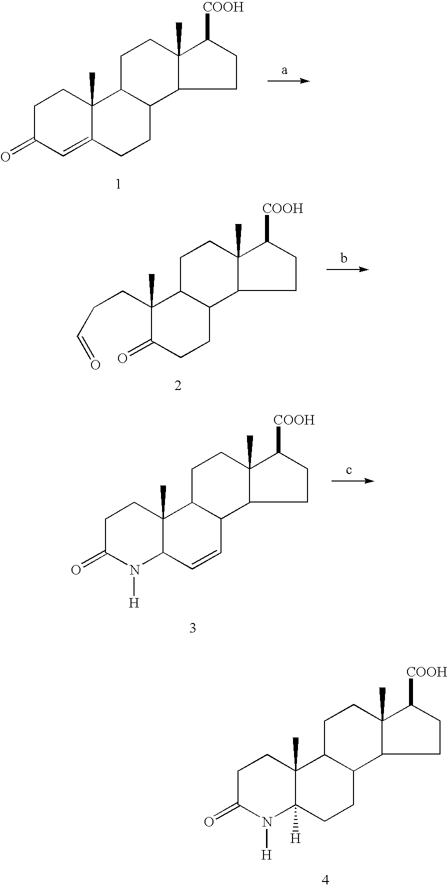
wherein: “a” indicates KMnO₄, NaIO₄, t-BuOH, reflux; “b” indicates NH₃, heat; and “c” indicates H₂, Pt, ArOH.

### SUMMARY OF THE INVENTION

An aspect of the present invention provides process for preparing a compound of Formula (I), which comprises:

(a) selective oxidation of a compound of Formula (II): 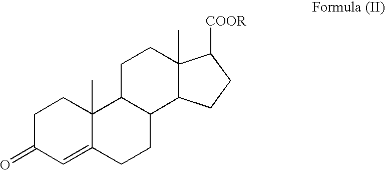
in which R is a C₁₋₅ branched or unbranched alkyl group, with an oxidizing agent to produce a compound of Formula (III): 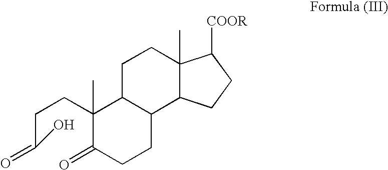
in which R is as defined in Formula (II);

(b) cyclization of the Formula (III) compound using a cyclizing agent to produce a compound of Formula (IV): 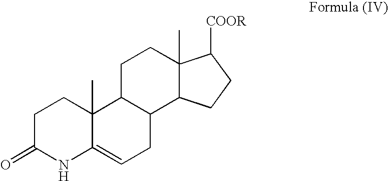
wherein R is as defined in Formula (II);

(c) reacting the compound having Formula (IV) with a reducing agent to yield a compound having Formula (V): 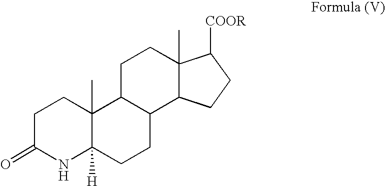
wherein R is as defined in Formula (II); and

(d) hydrolysis of the ester group of the compound of Formula (V) to produce the compound of Formula (I).

### BRIEF DESCRIPTION OF THE DRAWINGS

FIG. 1 shows a Powder X-ray diffraction pattern of 3-oxo-4-aza-5α-androstane-17β-carboxylic acid of Formula (I).

FIG. 2 shows an IR spectrum of 3-oxo-4-aza-5α-androstane-17β-carboxylic acid of Formula (I).

FIG. 3 is a schematic representation of a process for the preparation of 3-oxo-4-aza-5α-androstane-17β-carboxylic acid of Formula (I).

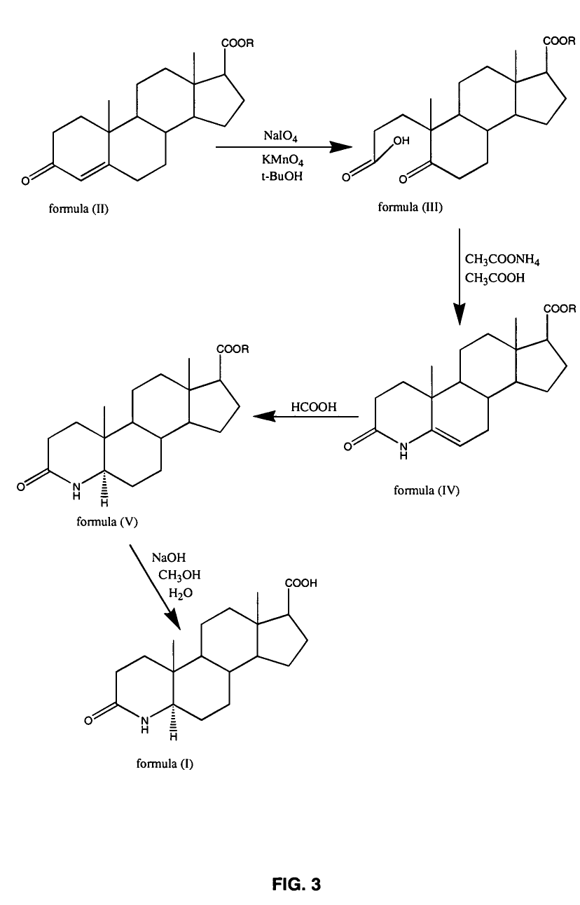

### DETAILED DESCRIPTION

An aspect of the present invention provides process for preparing a compound of Formula (I), which process comprises:

(a) selective oxidation of a compound of Formula (II): 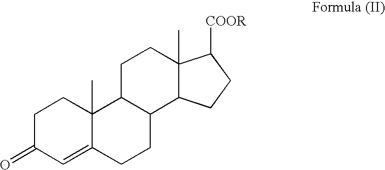
in which R is a C₁₋₅ branched or unbranched alkyl group, with one or more oxidizing agents in a solvent to produce a compound of Formula (III): 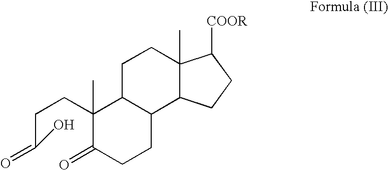
in which R is a C₁₋₅ branched or unbranched alkyl group;

(b) Cyclization of the Formula (III) compound with a suitable cyclizing agent to produce a compound of Formula (IV): 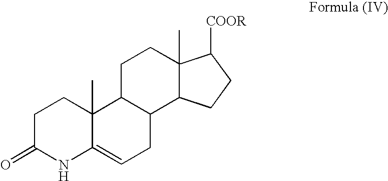
wherein R is as defined in Formula (II);

(c) reduction of the double bond in Formula (IV) to yield a compound having Formula (V): 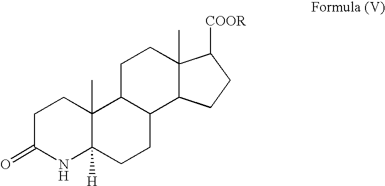
wherein R is as defined in Formula (II); and

(d) hydrolysis of the ester group of the compound of Formula (V) with a suitable basic hydrolyzing agent in an aqueous alcoholic solvent to produce the compound of Formula (I).

Step (a) involves selective oxidation of a compound of formula (II) in which R is a C₁₋₅ branched or straight chain alkyl group, to produce a compound of formula (III) in the presence of an oxidizing agent in a suitable solvent. Oxidation can be carried out by dissolving a compound of formula II in alcohol or ketonic solvents such as t-butanol or acetone in an amount of about 5-20 times by volume, or about 7-10 times by volume with respect to the compound of formula (II), prior to the addition of an inorganic base like sodium carbonate or potassium carbonate dissolved in a protic solvent such as water in the ratio of about 1:3 molar equivalents or about 1:2 molar equivalents, then adding an oxidizing agent or a mixture of oxidizing agents such as permanganate salts, including potassium permanganate, periodate salts, including sodium metaperiodate, and the like, optionally dissolved in a protic solvent such as water in the ratio of about 10:20 (v/v).

In some instances, it may be advantageous to add oxidizing agent in more than one portion. A first portion of the oxidizing agent can be added at an ambient temperature and the mixture maintained for at an elevated temperature, such as about 60-80° C., until the reaction is complete. A second portion of oxidizing agent can contain about 0.05-1.5 molar equivalents of potassium permanganate, or about 0.08-1 molar equivalents, and about 2-6 molar equivalents of sodium metaperiodate, or about 5 molar equivalents with respect to a compound of formula (II). The reaction mass is stirred until reaction is complete at an elevated temperature such as about 60-80° C., or about 60-70° C., then is allowed to cool to about 20 to 50° C. or about 30-40° C. The formed solid is separated by filtration and washed with a protic solvent such as water. A halogenated solvent such as chloroform, dichloromethane, dichloromethane and a protic solvent such as water is added to the filtrate, followed by adjusting the pH to around 2 to 3 with conc. hydrochloric acid at 0 to 10° C., separating the organic and aqueous layers and extracting the aqueous layer with a halogenated solvent such as dichloromethane followed by washing the organic layer with 2.5 to 4% aqueous sodium metabisulfite solution. The organic layer solvent can be removed by means of techniques like distillation or evaporation with or without applying reduced pressure.

A hydrocarbon solvent such as petroleum ether can be added to the above reaction mass followed by distilling of the solvent using techniques like distillation or evaporation, either by applying reduced pressure or without applying reduced pressure, to afford the compound of Formula (III).

Step (b) involves cyclization of the compound having Formula (III) to produce a compound of Formula (IV) in presence of a cyclizing agent in a suitable solvent. Cyclization of Formula (III) can be carried by adding cyclizing agent such as ammonium acetate in presence of acid such as acetic acid and refluxing, such as for about 1 to 5 hours. The reaction mass is then allowed to cool to about 35 to 45° C. or 25 to 35° C., a protic solvent such as water is added and the solid is separated, washed with a protic solvent such as water, followed by drying the obtained solid at 60 to 90° C. or 70 to 80° C. to afford the compound of Formula (IV).

Step (c) involves reduction of the compound of Formula (IV) to produce a compound of Formula (V) in the presence of a suitable reducing agent. The reduction can be carried out with reducing agents like formic acid, or H₂ in the presence of palladium, or H₂ in the presence of Raney nickel. The formic acid is used in an amount that is about 3-8 times or about 5 times the molar amount of the compound of Formula (IV). The reaction mixture is heated to temperatures of about 85 to 125° C., or 100 to 110° C. until the reaction is complete, such as for about 8 to 20 hours or 10 to 15 hours. The reaction mass can be allowed to cool to about 20 to 40° C., or about 25 to 35° C., and stirred as solids form, such as for about 0.5 to 3 hours or 1 to 2 hours, then the obtained solid is filtered and washed with water and the solid is dried at 60 to 110° C., or 70 to 100° C., to afford the compound of Formula (V).

Step (d) involves hydrolysis of the ester group of the compound of formula (V) to produce the compound of formula (I) in the presence of a suitable basic hydrolyzing agent and an aqueous or aqueous alcoholic solvent. The ester group of formula (V) can be hydrolyzed by adding a solution of base such as sodium hydroxide, potassium hydroxide, potassium t-butoxide, or sodium methoxide in an aqueous solvent, or aqueous alcoholic solvent such as methanol, ethanol, propanol, butanol and the like to a compound of formula (V), and heating the reaction mass to reflux for sufficient time to complete the reaction, such as for 1 to 7, or 2 to 5 hours. The hot reaction mass is allowed to cool to about 5 to 20° C., or 10 to 15° C. The pH then is usually adjusted to about 1 to 3, or about 2, with acids like hydrochloric acid, sulfuric acid, acetic acid etc., followed by filtration and drying the solid at a temperature of about 60 to 100° C. or 70 to 90° C. to afford the compound of formula (I) with an expected yield of around 85 to 98%.

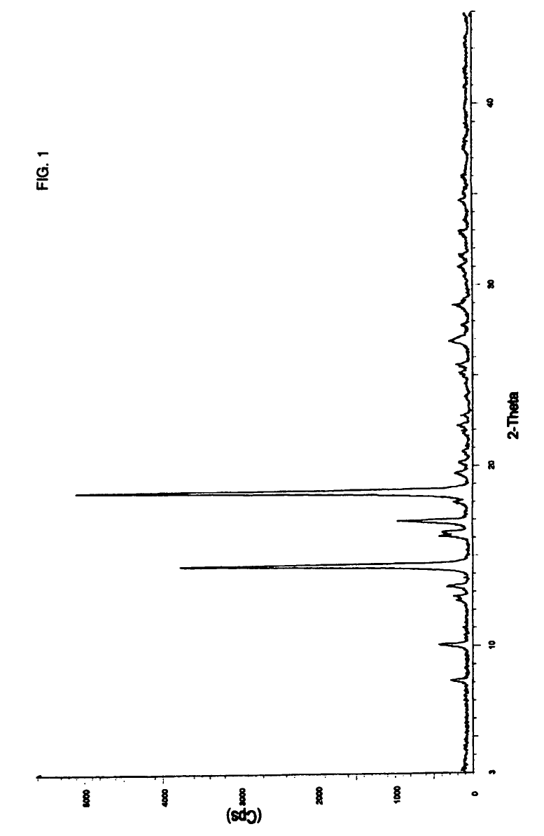

3-oxo-4-aza-5α-androstane-17β-carboxylic acid of formula (I) prepared according to the present process has the X-Ray powder diffraction pattern of FIG. 1, which was obtained using Cu Kα₁ radiation (1.541 Å). The more characteristic peaks of the X-Ray diffraction pattern, expressed as 2θ angles, are at about 8.0, 10.0, 12.7, 14.5, 16.1, 16.2, 16.9, 18.6, and 26.9±0.1 degrees.

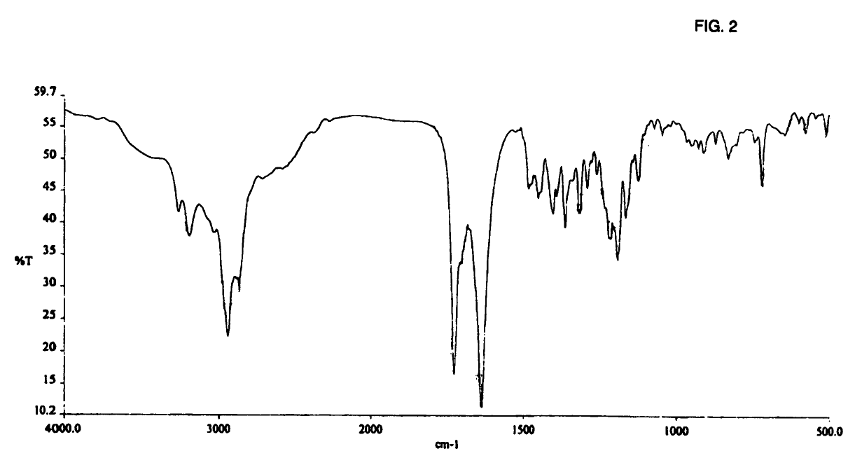

3-oxo-4-aza-5α-androstane-17β-carboxylic acid of formula (I) prepared according to the present process has the infrared absorption spectrum of FIG. 2, obtained by dispersing the sample in KBr. The more prominent peaks of the infrared spectrum were located at about 722, 831, 1121, 1190, 1289, 1360, 1401, 1482, 1637, 1728, 2869, 2942, 3031, 3193, and 3261 cm⁻¹. The location of peaks can show some variation, due to differences between analytical instruments, but the relationships of the peaks to each other will remain fairly constant.

The process of the present invention is simple, cost-effective and industrially feasible.

The present invention will be further explained using the following examples. However, it should be understood that the following examples are intended only to illustrate certain aspects of the present invention but not in any manner to limit the scope of the present invention.

### EXAMPLE 1

### SYNTHESIS OF 5-OXO-A-NOR-3,5-SECOAN DROANDROSTAN-17-CARBOXYLATE

50 grams of methyl-3-oxo-4-androstene-17-carboxylate was dissolved in 500 ml of t-butanol. Added a solution of 28 grams of sodium carbonate in 200 ml of water to the reaction mass. Added slowly 200 ml of a solution of 2 grams of sodium metaperiodate and 130 gram of potassium permanganate dissolved in 1250 ml of water at temperature of 25-35° C. for 30 minutes. Heated the reaction mixture to 65° C. and added remaining solution of sodium metaperiodate and potassium permanganate over one hour. The resulting solution was cooled to 40° C., filtered to remove the unwanted salts, and washed with water. 500 ml of dichloromethane and 500 ml of water were added to the filtrate. pH was adjusted to around 2 with 45 ml of concentrated hydrochloric acid at the temperature of 0° C. and aged the reaction mass at 0° C. for 30 minutes. Aqueous and organic layers were separated and the aqueous layer was extracted with 750 ml of dichloromethane. Washed the total organic layer with 500 ml of 3.5% aqueous solution of sodium metabisulfite solution and followed by 500 ml of water. Distilled off the solvent under a reduced pressure and 140 ml of petroleum ether was added to the residue. Distilled off the solvent completely to obtain the title compound as a residue. (Weight: 51-53 grams).

### EXAMPLE 2

### SYNTHESIS OF METHYL-3-OXO-4-AZA-5-ANDROSTENE-17-CARBOXYLATE

175 ml of acetic acid, 49 grams of ammonium acetate and 50 grams of methyl 5-oxo-A-nor-3,5-secoandroandrostan-17-carboxylate were stirred well and then the reaction mass was refluxed for 2-3 hours. Cooled the reaction mass to 25-35° C. 250 ml of water was added to the reaction mass and stirred for 2-3 hours. The separated solid was filtered and washed with 50 ml of water. Dried the solid at 70° C. to get the title compound (36.5 grams, 77.3% yield).

### EXAMPLE 3

### SYNTHESIS OF METHYL-3-OXO-4-AZA-5α-ANDROSTAN E-17-CARBOXYLATE

175 ml of formic acid was added to 35 gram of methyl-3-oxo-4-aza-5-androstene-17-carboxylate prepared in Example 2 and refluxed for 10-12 hours. Added the obtained reaction mass to 1750 ml of water slowly at 25-35° C. Stirred the reaction mixture for 2 hours at 25-35° C., filtered the obtained compound and washed with 1750 ml of water. Dried the compound at 90° C. to get 33.5 grams of title compound (Yield: 95%).

### EXAMPLE 4

### PURIFICATION OF METHYL-3-OXO-4-AZA-5α-ANDROSTANE-17-CARBOXYLATE

10 grams of methyl-3-oxo-4-aza-5α-androstane-17-carboxylate was suspended in 50 ml of dichloromethane and refluxed for 20-30 minutes. 150 ml of ethyl acetate was added and refluxed for 1-2 hours. The reaction mixture was cooled to 25-30° C. and the separated solid was filtered and washed with 20 ml of ethyl acetate. Dried the obtained solid at 70° C. to obtain 7.0 grams of the title compound.

### EXAMPLE 5

### SYNTHESIS OF 3-OXO-4-AZA-5α-ANDROSTANE-17-CARBOXYLIC ACID

60 ml of a 9% aqueous sodium hydroxide solution was added to 15 grams of methyl-3-oxo-4-aza-5α-androstane-17-carboxylate and refluxed for 3-4 hours. The reaction mass was cooled to 10-15° C. and pH was adjusted to around 2 with concentrated hydrochloric acid. Filtered the separated solid and washed with 75 ml of water, then dried at 80° C.

We claim:

1. A process for preparing a compound having Formula (I): 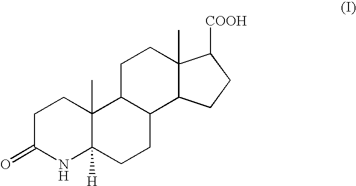
comprising:
(a) reacting a compound having Formula (II): 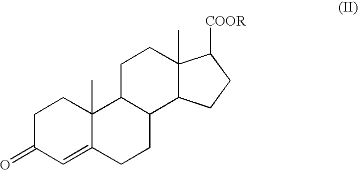
where R is a branched or unbranched C₁-C₅ alkyl group, with an oxidizing agent to form a compound having Formula (III): 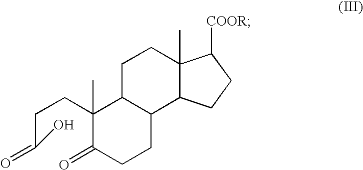
(b) reacting the compound having Formula (III) with a cyclizing agent to form a compound having Formula (IV): 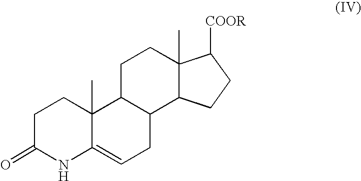
where R is as defined above; and
(c) reacting the compound having Formula (IV) with a reducing agent to form a compound having Formula (V): 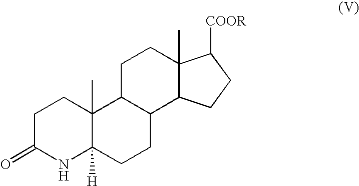
where R is as defined above; and
(d) hydrolyzing the compound having Formula (V) to form the compound having Formula (I).

2. The process of claim 1, wherein the oxidizing agent comprises a permanganate salt, a periodate salt, or a mixture thereof.

3. The process of claim 1, wherein oxidizing agent is added in multiple portions.

4. The process of claim 1, wherein the cyclizing agent comprises ammonium acetate in the presence of acetic acid.

5. The process of claim 1, wherein the reducing agent comprises formic acid.

6. The process of claim 1, wherein hydrolyzing occurs under basic conditions.

7. A process for preparing a compound having formula (I): 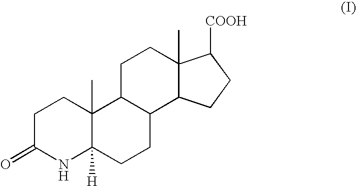
comprising:
(a) reacting a compound having Formula (II): 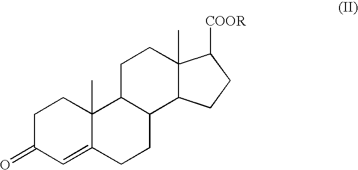
where R is a branched or unbranched C₁-C₅ alkyl group, with an oxidizing agent comprising potassium permanganate and sodium metaperiodate to form a compound having Formula (III): 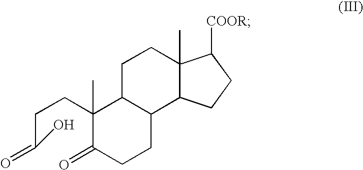
(b) reacting the compound having Formula (III) with a cyclizing agent comprising ammonium acetate and acetic acid to form a compound having Formula (IV): 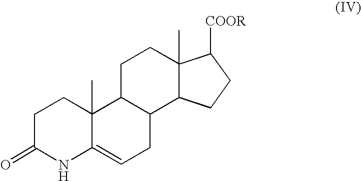
where R is as defined above; and
(c) reacting the compound having Formula (IV) with a reducing agent comprising formic acid to form a compound having Formula (V): 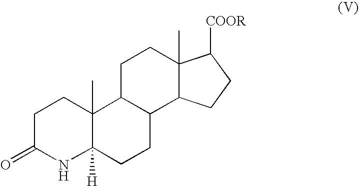
where R is as defined above; and
(d) hydrolyzing the compound having Formula (V) with a base to form the compound having Formula (I).
